(19) United States
(12) Patent Application Publication
(10) Pub. No.: US 2012/0065627 A1
(43) Pub. Date: Mar. 15, 2012

(54) Non-Clumping Unit For Use With A Magnetic Surgical System

(76) Inventors: Ragae M. Ghabrial, Loveland, OH (US); James T. Spivey, Cincinnati, OH (US); Christopher J. Hess, Cincinnati, OH (US); Sean P. Conlon, Loveland, OH (US)

(21) Appl. No.: 12/879,310

(22) Filed: Sep. 10, 2010

(51) Int. Cl. A61B 17/00

(52) U.S. Cl. 606/1

(57) ABSTRACT

A surgical device comprises an ex vivo magnet and an in vivo sled magnetically attracted to the ex vivo magnet. The sled can be positioned and anchored within a patient by moving the ex vivo magnet. The sled defines a longitudinal axis. An arm extends from the sled. The arm is moveable relative the sled between a retracted position and an extended position. The arm comprises an end effector. A locking mechanism operatively connected to the arm to lock the arm in the retracted and extended positions. The mechanism may include a rack that both rotates about a pinion and translates tangentially about the pinion.

### BACKGROUND

The present invention relates in general to surgical devices and procedures, and more particularly to minimally invasive surgery.

Surgical procedures are often used to treat and cure a wide range of diseases, conditions, and injuries. Surgery often requires access to internal tissue through open surgical procedures or minimally invasive surgical procedures. Minimally invasive surgery often involves using an endoscope, such as laparoscopes, arthroscopes, and flexible endoscopes, to visualize internal tissue of a patient, which sometimes referred to as “endoscopic surgery”. Endoscopes and instruments are typically introduced into a patient through percuateous punctures or incisions, or through a patient's natural orifices to access intraluminal anatomy or for transluminal procedures.

Minimally invasive surgery has numerous advantages compared to traditional open surgical procedures, including reduced trauma, faster recovery, reduced risk of infection, and reduced scarring. Minimally invasive surgery is often performed with an insufflatory fluid present within the body cavity, such as carbon dioxide or saline, to provide adequate space to perform the intended surgical procedures. The insufflated cavity is generally under pressure and is sometimes referred to as being in a state of pneumoperitoneum. Surgical access devices are often used to facilitate surgical manipulation of internal tissue while maintaining pneumoperitoneum. For example, trocars may be used to provide a port through which endoscopes and surgical tools are passed. Trocars generally have an instrument seal, which prevents the insufflatory fluid from escaping while an endoscope or surgical instrument is positioned in the trocar.

Minimally invasive surgery may also be performed using magnetic anchors that enable positioning and manipulation of surgical tools that are introduced into a patient's abdominal cavity through percutaneous punctures or incisions. Using magnetic anchors may reduce the number of trocars needed in a particular procedure and in turn reduce the number of incisions to the patient's body.

### BRIEF DESCRIPTION OF DRAWINGS

While the specification concludes with claims which particularly point out and distinctly claim the invention, it is believed the invention will be better understood from the following description taken in conjunction with the accompanying drawings illustrating some non-limiting examples of the invention. Unless otherwise indicated, like-numbered references refer to the same elements in the various figures. Unless otherwise indicated, the figures are not necessarily drawn to scale, but rather to illustrate the principles of the invention.

FIG. 1 depicts a cross-sectional view of a non-clumping unit engaging two ex vivo magnets;

FIG. 2 depicts an isometric view of a non-clumping unit engaging two ex vivo magnets;

FIG. 3 depicts an isometric view of a non-clumping unit with a pivoting pin engaging two ex vivo magnets;

FIG. 4 depicts an isometric view of a non-clumping unit with a pivoting pin;

FIG. 5 depicts an isometric view of a non-clumping unit engaging two ex vivo magnets;

FIG. 6 depicts an isometric view of a non-clumping unit with a ball and socket joint;

FIG. 7 depicts an isometric view of a non-clumping unit engaging two ex vivo magnets;

FIG. 8 depicts an isometric view of a non-clumping unit with a track;

FIG. 9 depicts an isometric view of an ex vivo magnet enclosed in a non-magnetic housing;

FIG. 10 depicts a cross sectional view of a an ex vivo magnet enclosed in a non-magnetic housing; and

FIG. 11 depicts an isometric view of two ex vivo magnets equipped with an ultrasonic transmitter and a receiver.

### SUMMARY

In one embodiment, a surgical device comprises a first and a second ex vivo magnets adapted for manipulation of in vivo instruments. A separator has a first end, a second end, and an elongate body extending between the first and second ends. A first connector at the first end releasably engages the first ex vivo magnet, and a second connector at the second end of the elongated body releasably engages the second ex vivo magnet. The elongated body may be fully rigid. Alternatively it may comprise a locking flexible section with alternating rigid and flexible segments.

The first connector may comprise a ball and a socket joint to facilitate the movement of the connector relative to the elongated body. The first connector may also comprise a pivot pin. The first connector may comprise a track for operably receiving a mating element at the first end of the elongated body. The first connector may be in the shape of a ring that circumscribes the first ex vivo magnet, and may allow the ex vivo magnet to rotate relative to it.

In another embodiment, a surgical device comprises an ex vivo magnet and an in vivo magnet attracted to the ex vivo magnet, whereby the in vivo magnet can be positioned and anchored within a patient by moving the ex vivo magnet. A non-magnetic housing substantially encloses the ex vivo magnet and has a wall thickness sufficient to minimize attraction between the ex vivo magnet and other ex vivo magnets. The ex vivo magnet may be fully enclosed in the non-magnetic housing.

In yet another embodiment, a surgical device comprises an ex vivo magnet, an in vivo magnet attracted to the ex vivo magnet, whereby the in vivo magnet can be positioned and anchored within a patient by moving the ex vivo magnet A sensor detects the relative distance between the ex vivo magnet and another ex vivo magnet. An an indicator warns an operator that further movement of the ex vivo magnet in the direction of the other ex vivo magnet may cause clumping. In one embodiment, the sensor is an ultrasonic sensor. The indicator may be a light indicator. It may also be a sound indicator.

### DETAILED DESCRIPTION

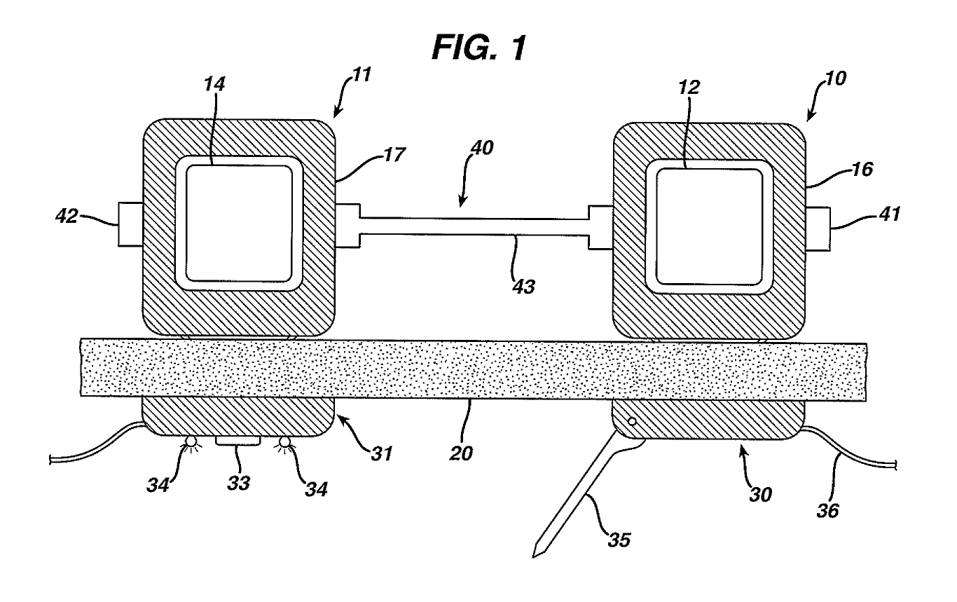

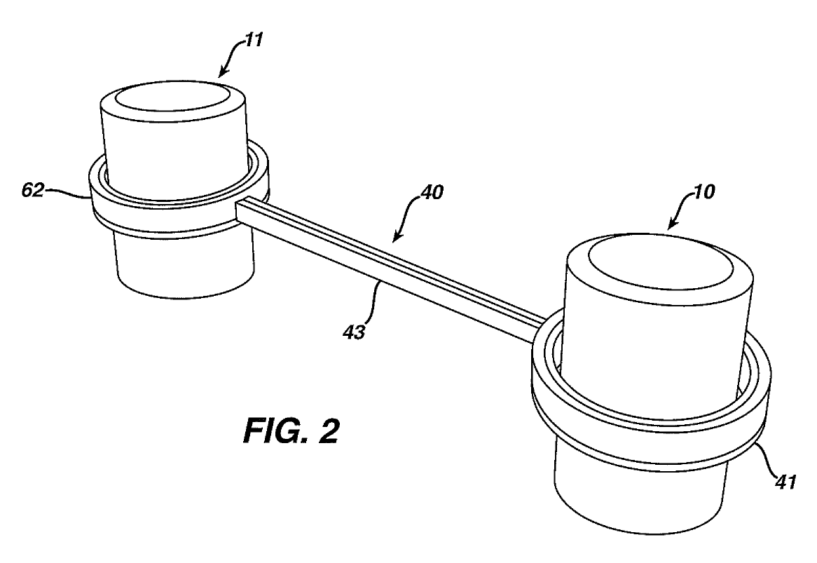

The embodiment shown in FIGS. 1-2 comprises two anchors (10, 11) and two sled bases (30, 31). Patient tissue (20), such as the abdominal wall, an organ wall, or the like, is interposed between the anchor (10, 11) and the sled bases (30, 31). The anchors (10, 11) are magnetically coupled to the sled bases (30, 31) respectively through the tissue (20). By sliding the anchor (10) relative the tissue (20), the surgeon can position the sled base (30) in a desired location inside the patient's cavity. Likewise, by keeping the anchor (11) stationary relative the tissue (20), the surgeon can anchor the sled base (31) in a desired location. The anchors (10, 11) will often be positioned ex vivo and the sled bases (30, 31) positioned in vivo.

The anchor (10) includes a magnet (12). The magnet (12) is contained within a casing (16) that forms an ergonomic handle. The anchor (11) includes a magnet (14) contained within a casing (17). The magnets (12, 14) can take a variety of forms such as permanent magnets, rare earth magnets, electromagnets, and the like.

In the present embodiment, a non-clumping unit (40) is used to prevent the anchors (10, 11) from clumping together during a surgical procedure. The attractive forces between the anchors (10, 11) increase as the distance separating them decreases. The term clumping describes two or more magnetic anchors being magnetically coupled to one another. Clumping may occur during a surgical procedure if the surgeon positions the anchors (10, 11) in close proximity to each causing the attractive forces between the anchors (10, 11) to overcome the forces keeping them stationary. Sometimes clumping can occur through a rapid clashing of the magnets to one another. Once clumped together, the attractive forces between anchors (10, 11) are at their highest and separating the anchors (10, 11) may prove difficult.

In the present embodiment, the non-clumping unit (40) comprises two ring shaped connectors (41, 42) where the connectors (41, 42) are fixed at opposite ends of an elongated bar (43). The anchors (10, 11) are releasably coupled to connectors (41, 42). Anchors (10, 11) may be freely rotated each around its central axis while coupled to the connectors (41, 42). The connectors (41, 42) may be rigid or flexible.

Elongated bar (43) is of sufficient length to ensure that anchors (10, 11) remain a suitable distance apart from each other such that a surgeon can operate each freely without any significant interference caused by the attractive forces between the anchors (10, 11). For example, two identical anchors comprising 2 inch cube neodymium magnets should be kept a minimum distance of 4-6 cm apart at all times during a surgical procedure to reduce the risk of clumping. Preferably the anchors should be kept 6 cm apart. In this example, a non-clumping unit with an elongated bar having a length of about 6 cm may be used to allow the surgeon to operate the anchors freely without constantly worrying about clumping.

Use of multiple anchors may be preferable in a surgical procedure as it permits the surgeon to utilize multiple sled bases. The sled bases (30, 31) are sized to pass through a standard trocar, such as a 12 mm, 18 mm, or 20 mm trocar. A slid base may comprise a camera, a light source, or a surgical end effector. A variety of end effectors could be used, including graspers, scissors, ultrasonic blades, bi-polar clamps, surgical staplers, ultrasonic sensors, cameras, suturing devices, and the like. In this embodiment, the sled base (31) comprises a wireless camera (33) and LEDs (34). The sled base (30) comprises a surgical cautery end effector (35). A wire (36) is operatively connected to the surgical caurtery end effector (35) and extends from the sled base (30) to deliver electrical energy.

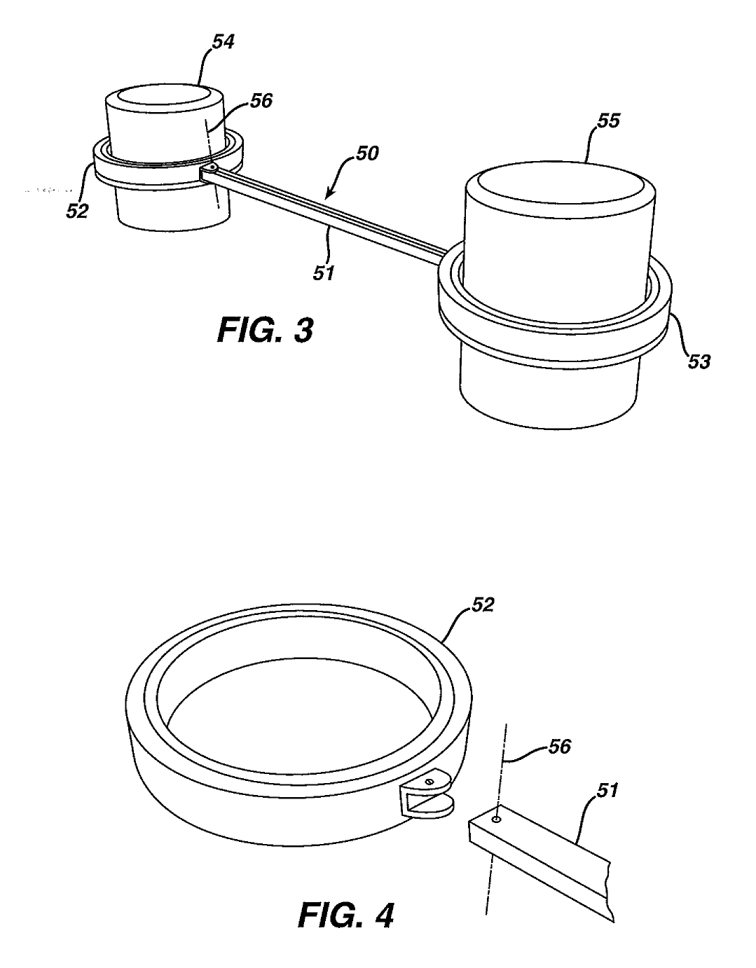

FIGS. 3-4 show a non-clumping unit (50) comprising an elongated bar (51) and two connectors (52, 53) releasably coupled to anchors (54, 55). The connector (52) pivots relative to elongated bar (51) about the pivot axis (56) which gives the surgeon more freedom in positioning anchor (54) at an angle with elongated bar (51) while maintaining a suitable distance between anchors (54, 55).

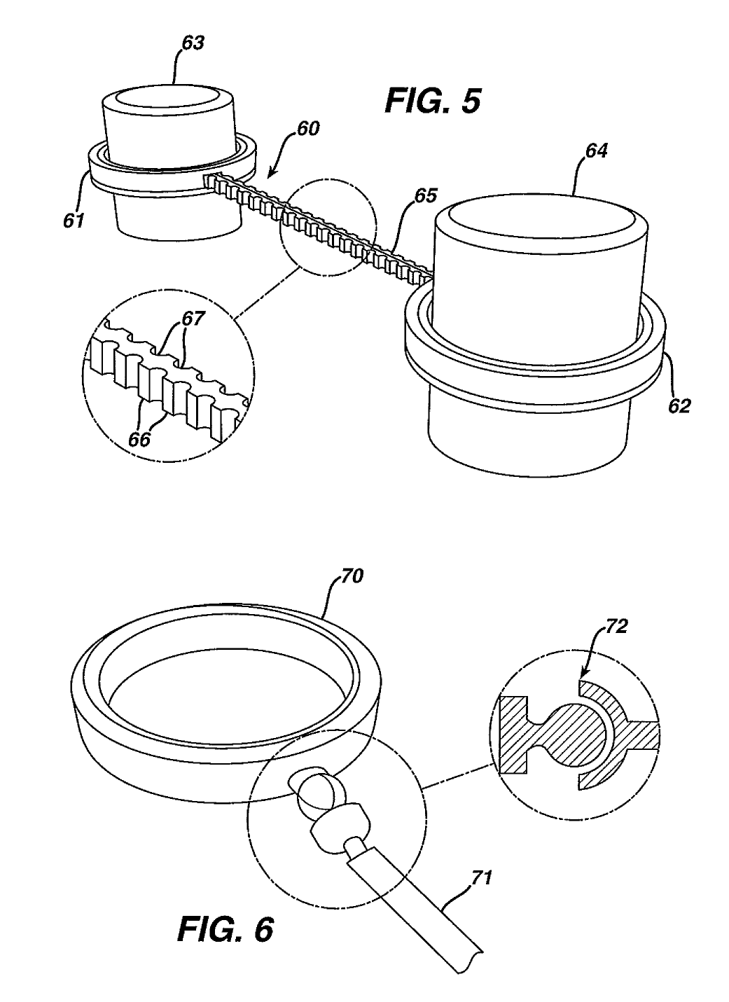

FIG. 5 shows a non-clumping unit (60) that comprises two connectors (61, 62) which are releasably coupled to anchors (63, 64). The connectors (61, 62) are fixed at opposite ends of an elongated bar (65). In this embodiment, the elongated bar (65) comprises several alternating rigid (66) and flexible (67) segments. The flexible segments (67) allow the surgeon to freely move the anchors (63, 64) relative to each other by bending the elongated bar (65). In a fully bent position, the elongated bar (65) forms an arc with the rigid segments (66) in full contact with each other which prevents the elongated bar (65) from any further bending. The distance between the anchors (63, 64) while the elongated bar (65) is in a fully bent position is sufficiently long to prevent clumping.

FIG. 6 shows a connector (70) linked to an elongated bar (71) via a ball and socket joint (72). In this embodiment, the ball and socket joint (72) provides the surgeon with more flexibility in positioning a magnetic anchor enclosed by connector (70) at an angle with the elongated bar (71).

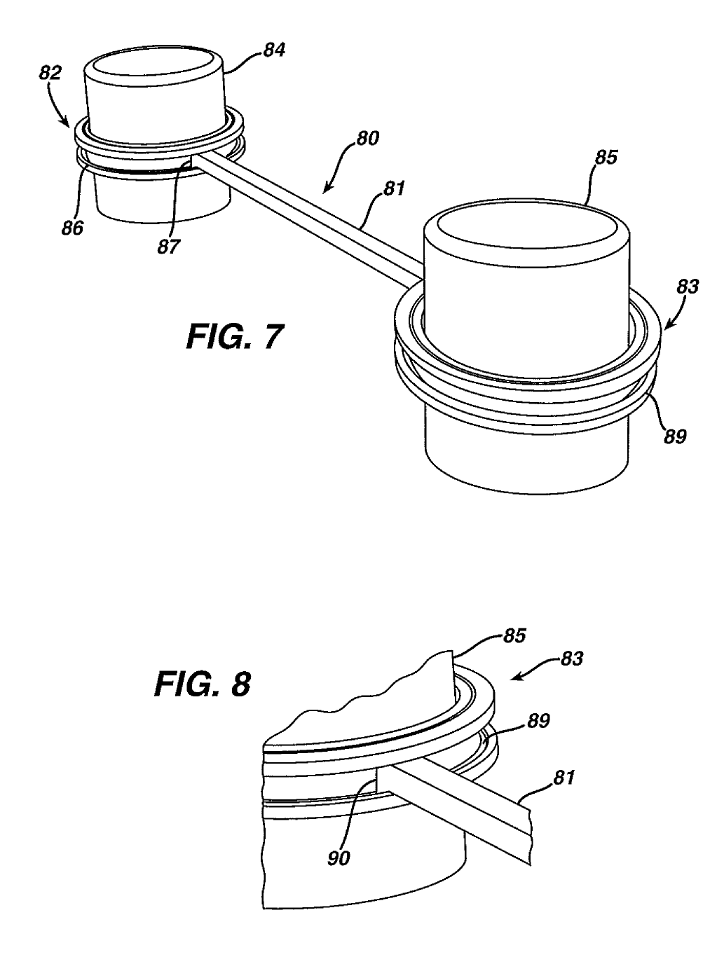

FIGS. 7-8 show a non-clumping unit (80) comprising an elongated bar (81) and two connectors (82, 83) releasably coupled to magnetic anchors (84, 85). The Connector (82) comprises a track (86) which operably receives a mating element (87) fixed at one end of the elongated bar (81). The connector (83) comprises another track (89) which operably receives another mating element (90) fixed at the opposite end of the elongated bar (81). In this embodiment, the connectors (82, 83) are snapped onto opposite ends of the elongated bar (81) which facilitates exchanging anchors during a surgical procedure. An anchor fitted with a connector can be readily snapped into position.

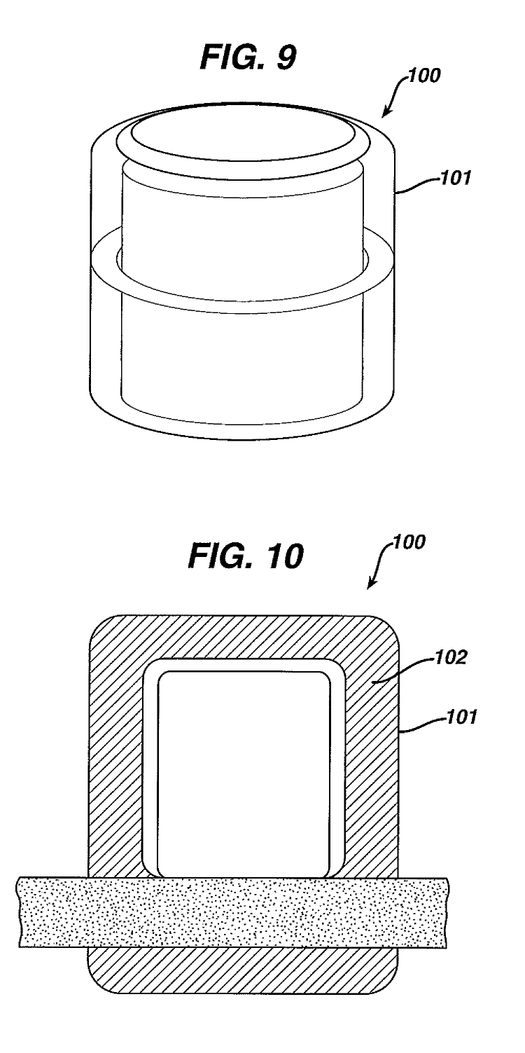

FIGS. 9-10 show a magnetic anchor (100) in a non-magnetic housing (101). The non-magnetic housing wall (102) is of sufficient thickness to prevent clumping between the magnetic anchor (100) and other magnetic anchors that may be used by the surgeon. In this embodiment, the non-magnetic housing (101) is dropped over the magnetic anchor (100) prior to introduction of other magnetic anchors into the operating field. For example, a surgeon may start the procedure by using a first magnetic anchor for positioning a camera. Once the camera is in position, a non-magnetic housing is dropped over the anchor. Then another magnetic anchor is introduced into the operating field which can be used by the surgeon to manipulate an end effector such as a surgical cautery end effector.

In yet another embodiment, magnetic anchors in a surgical procedure can be equipped with sensors to measure the relative distance between them. A controller may be used to process the information and signal an indicator when the magnetic anchors are too close to each other. A light or a sound indicator can be used to warn an operator that the anchors are too close to each other which protects against clumping.

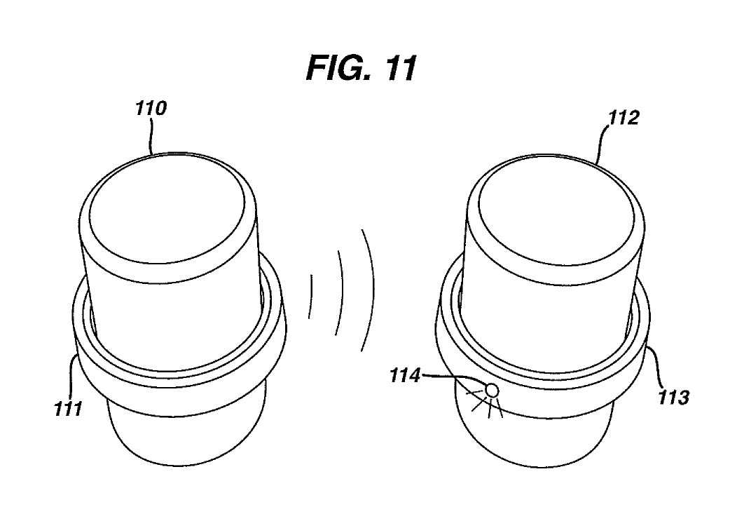

FIG. 11 shows a magnetic anchor (110) equipped with an ultrasonic transmitter (111). Another magnetic anchor (112) is equipped with an ultrasonic detector (113), a controller and a light indicator (114). Ultrasonic transmitter (111) generates a high frequency sound wave. Ultrasonic detector (113) receives the sound wave and calculates the time interval between sending the signal and receiving it which is then translated into distance. Light Indicator (114) lights up to warn the operator if anchors (110, 112) are positioned at a distance less than a minimum distance recognized by the controller.

Having shown and described various embodiments and examples of the present invention, further adaptations of the methods and devices described herein can be accomplished by appropriate modifications by one of ordinary skill in the art without departing from the scope of the present invention. Several of such potential modifications have been mentioned, and others will be apparent to those skilled in the art. For instance, the specific materials, dimensions, and the scale of drawings will be understood to be non-limiting examples. Accordingly, the scope of the present invention should be considered in terms of the following claims and is understood not to be limited to the details of structure, materials, or acts shown and described in the specification and drawings.

1. A surgical device comprising:
a) a first ex vivo magnet adapted for manipulation of in vivo instruments;
b) a second ex vivo magnet adapted for manipulation of in vivo instruments;
c) a separator having a first end, a second end, and an elongate body extending between the first and second ends;
d) a first connector at the first end of the elongated body for engaging the first ex vivo magnet; and
e) a second connector at the second end of the elongated body for engaging the second ex vivo magnet.

2. A surgical device of claim 1, wherein the first connector releasably engages the first ex vivo magnet.

3. A surgical device of claim 2, wherein the second connector releasably engages the second magnet.

4. The surgical device of claim 1, wherein the first connector comprises a ball and a socket joint.

5. The surgical device of claim 1, wherein the first connector comprises a pivot pin.

6. The surgical device of claim 1, wherein the elongated body comprises a locking flexible section.

7. The surgical device of claim 1, wherein the first connector comprises a track for operably receiving a mating element at the first end.

8. The surgical instrument of claim 1, wherein the first connector comprises a ring that circumscribes the first ex vivo magnet.

9. The surgical device of claim 8, wherein the first ex vivo magnet can rotate relative the ring.

10. A surgical device, comprising:
a) an ex vivo magnet;
b) an in vivo magnet attracted to the ex vivo magnet, whereby the in vivo magnet can be positioned and anchored within a patient by moving the ex vivo magnet; and
c) a non-magnetic housing substantially enclosing the ex vivo magnet, wherein the non-magnetic housing has a wall thickness sufficient to minimize attraction between the ex vivo magnet and other ex vivo magnets.

11. The surgical device of claim 9, wherein the ex vivo magnet is fully encased in the non-magnetic housing.

12. The surgical device of claim 9, wherein the non-magnetic housing is made from an elastomeric material.

13. The surgical device of claim 9, wherein the ex vivo magnet is releasably held in the non-magnetic housing.

14. A surgical device, comprising:
a) an ex vivo magnet;
b) an in vivo magnet attracted to the ex vivo magnet, whereby the in vivo magnet can be positioned and anchored within a patient by moving the ex vivo magnet;
c) a sensor for detecting the relative distance between the ex vivo magnet and another ex vivo magnet; and
d) an indicator for warning an operator that further movement of the ex vivo magnet in the direction of the other ex vivo magnet may cause clumping.

15. The surgical, device of claim 14, wherein the sensor is an ultrasonic sensor.

16. The surgical device of claim 14, wherein the indicator is a light indicator.

17. The surgical device of claim 14, wherein the indicator is a sound indicator.
